(19) United States
(12) Patent Application Publication
(10) Pub. No.: US 2010/0152725 A1
(43) Pub. Date: Jun. 17, 2010

(54) METHOD AND SYSTEM FOR TISSUE TREATMENT UTILIZING IRREVERSIBLE ELECTROPORATION AND THERMAL TRACK COAGULATION

(75) Inventors: Robert M. Pearson, San Jose, CA (US); Eamonn P. Hobbs, Queensbury, NY (US); William M. Appling, Granville, NY (US)

Correspondence Address: ANGLODYNAMICS, INC., 14 PLAZA DRIVE, LATHAM, NY, 12110, US

(73) Assignee: AngioDynamics, Inc., Queensbury, NY (US)

(21) Appl. No.: 12/627,008

(22) Filed: Nov. 30, 2009

Related U.S. Application Data
(60) Provisional application No. 61/122,058, filed on Dec. 12, 2008.

(51) Int. Cl. A61B 18/14; A61B 18/18

(52) U.S. Cl. 606/33; 606/41

(57) ABSTRACT

A system for selectively ablating tissue is provided herein that has at least one energy source that has a non-thermal energy source and a thermal energy source, at least one probe, means for selectively coupling the probe to one desired energy source of the at least one energy source, means for selectively energizing the non-thermal energy source of the at least one energy source to apply non-thermal energy to at least a portion of the desired region to ablate at least a portion of the desired region, and means for selectively energizing the thermal energy source of the at least one energy source during the withdrawal of the at least one probe to thermally ablate tissue substantially adjacent to a probe track.

### CROSS REFERENCE TO RELATED APPLICATIONS

This application claims priority to U.S. Provisional Application Ser. No. 61/122,058, filed Dec. 12, 2008, which is incorporated herein by reference.

### FIELD OF THE INVENTION

The present invention relates to systems and methods for treating target regions of tissue. More particularly, the present invention relates to a combination system and method for non-thermally treating target regions of tissue and subsequently thermally ablating tissue along a tissue track to coagulate blood vessels and prevent track seeding.

### BACKGROUND

Tumor ablation technology for medical treatment is known in the art and includes such treatment modalities as radiofrequency (RF), focused ultrasound, such as high intensity ultrasound beams, microwave, laser, thermal electric heating, traditional heating methods with electrodes using direct current (DC) or alternating current (AC), and application of heated fluids and cold therapies (such as cryosurgery, also known as cryotherapy or cryoablation).

In many of these procedures an energy delivery device, such as a probe with or without a needle, is inserted into a target tissue to cause destruction of a target region of a tumor mass through the application of energy, such as thermal energy, non-thermal energy, and energy associated with cryo ablation procedures. Upon insertion of the energy delivery device, a tissue track is typically created. A tissue track is defined as the space created by the insertion of a device extending from the skin surface to the target tissue. When the energy delivery device is removed, it is pulled back along the tissue track that had been previously created upon insertion of the energy delivery device. As the energy delivery device is being withdrawn, the tissue immediately adjacent to the puncture site or tissue track is ablated. The settings for the track ablation procedure can be significantly lower than the ablation settings necessary for non-tissue track ablation. This can produce a focalized zone around the tissue track, maximizing the chance of death of unwanted cellular material that may have adhered to the energy delivery device, thereby preventing undesirable cell displacement, such as in the movement of tumor cells that can re-seed to secondary locations. It is known in the art that electrically induced thermal ablation such as RF can be used to effectively and continuously locally ablate a tissue track as an energy delivery device is being removed to prevent tumor cell seeding and track bleeding. RF can lead to coagulation necrosis in a margin surrounding normal tissue where hyperthermic conditions lead to cellular injury such as coagulation of cytosolic enzymes and damage to histone complexes, leading to ultimate cell death. Although these tissue treatment methods and systems can effectively ablate volumes of target tissue, there are limitations to each technique. One often cited problem using these procedures during tumor ablation involves heat sink, a process whereby one aspect can include blood flow dragging thermal energy away from a target tissue. This heat sink effect can change both the shape and maximum volume of tissue that can be treated. After treatment of a target tissue region with an energy delivery device, upon removal of the energy delivery device from the targeted tissue region, tumor cells can be pulled back with the energy delivery device (i.e., seeding) along the tissue track or bleeding can occur along the tissue track.

More recently, irreversible electroporation (IRE) has been used as an alternative to the above-mentioned procedures to ablate tumor tissue. However through IRE can be a nonthermal method mediating cell death, it is not ideal for coagulation, and specifically does not cause electrically induced thermal coagulation, demonstrating the importance of using an alternative source such as RF or long DC pulses in heating a tissue track. Instead, IRE involves the application of electrical pulses to target tumor tissue in the range of microseconds to milliseconds that can lead to non-thermally produced defects in the cell membrane that are nanoscale in size. These defects can lead to a disruption of homeostasis of the cell membrane, thereby causing irreversible cell membrane permeabilization which induces cell necrosis, without raising the temperature of the tumor ablation zone. During IRE ablation, connective tissue and scaffolding structures are spared, thus allowing the surrounding bile ducts, blood vessels, and connective tissue to remain intact. With nonthermal IRE (hereinafter also called non-thermal IRE), cell death is mediated through a nonthermal mechanism, so the heat sink problem associated with many ablation techniques is nullified. Therefore the advantages of IRE to allow focused treatment with tissue sparing and without thermal effects can be used effectively in conjunction with thermal treatment such as RF that has been proven effective to prevent track seeding; this will also allow (in this example embodiment) the user to utilize determined RF levels leading to in some cases ablation and in some cases coagulation of blood vessels of all sizes encountered during treatment; this is important since IRE will not effectively coagulate when dealing with large vessels. In this way the newly discovered advantages of IRE can be utilized effectively with known techniques of thermal damage including mediating tumor cell death and bringing about coagulation along a tissue track.

Although IRE has distinct advantages, there are also advantages of utilizing thermal ablation during withdrawal of energy delivery devices from ablated tumor regions. Prior to the disclosure of this invention, an invention had not been proposed that could solve the problems of nonthermally ablating a target region of tumor tissue, while maintaining integrity of the surrounding tissue, and effectively switching to a device for effectively thermally ablating tissue along the probe track. In certain proposed embodiments, an energy delivery device can be utilized that is powered by a single energy source that is capable of application of energy in various forms, and subsequently ablating a tissue track during withdrawal of the same energy delivery device that can be powered by a different form of energy from the same energy source, to prevent track seeding and minimize bleeding. As indicated, IRE provides advantages for nonthermal cell death and thermal mechanisms provide advantages for not only preventing seeding, but also for effectively bringing about coagulation. A need exists for a system and method that can provide this combined non-thermal/thermal tumor ablation and that allows for switching between non-thermal IRE energy delivery and thermal energy delivery to increase tumor ablation efficiency and efficacy and the prevention of tissue track seeding.

It is a purpose of this invention, in certain embodiments, to provide a combination treatment system that has at least one energy delivery device and at least one power or energy or power source that is capable of providing IRE energy and thermal energy to the energy delivery device. The at least one energy delivery device can be either a monopolar or bipolar device. The system can have at least one manual or automatic switching device for switching the energy or power source from energy utilized in a nonthermal form to energy in a thermal form to ablate target tumor regions of tissue as well as tissue along a track.

It is a further purpose of this invention to provide a method that involves using non-thermal IRE energy and thermal energy to effectively ablate target regions of tissue. The method involves positioning at least one energy delivery device that is coupled to a single power source within a target region of a tissue, applying IRE energy from the power source to the energy delivery device which is used to ablate a target region of tissue, while preventing damage to surrounding structures, then switching from IRE energy to thermal energy using the same power source, and withdrawing the energy delivery device while ablating a tissue track with thermal energy such as RF energy, to allow for focal tissue ablation and the safe and efficient withdrawal of the energy delivery device used during the treatment procedure, while among other things, coagulating tissue and preventing track seeding.

### SUMMARY

What is described herein is a system and method for selectively ablating tissue. In certain embodiments the method involves providing application of IRE to treat tissue and treatment of tissue with an alternative energy form (such as thermal energy) to effectively ablate track tissue as a probe is withdrawn. The method can involve providing at least one energy source which has at least a non-thermal energy source and a thermal energy source, providing at least one probe that is configured to be selectively operatively coupled to a desired energy source of the at least one energy source, positioning via a probe track at least a portion of the at least one probe within a desired region of a target tissue, selectively coupling the at least one probe to the non-thermal energy source, selectively energizing the non-thermal energy source to apply non-thermal energy from the non-thermal energy source to at least a portion of the desired region to ablate at least a portion of the desired region, selectively coupling the at least one probe to the thermal energy source, withdrawing the at least probe from the desired region, and selectively energizing the thermal energy source to apply thermal energy during at least a portion of withdrawal of the at least one probe to ablate tissue substantially adjacent to the probe track.

A system for selectively ablating tissue is provided herein that has at least one energy source that has a non-thermal energy source and a thermal energy source, at least one probe, a means for selectively coupling the probe to one desired energy source of the at least one energy source, means for selectively energizing the non-thermal energy source of the at least one energy source to apply non-thermal energy to at least a portion of the desired region to ablate at least a portion of the desired region, and means for selectively energizing the thermal energy source of the at least one energy source during the withdrawal of the at least one probe to thermally ablate tissue substantially adjacent to a probe track.

### BRIEF DESCRIPTION OF THE SEVERAL VIEWS OF THE DRAWINGS

A more complete understanding of the present invention can be derived by referring to the detailed description when considered in connection with the following illustrative figures. In the figures, like reference numbers refer to like elements or acts throughout the figures. Elements and method steps illustrated in the figures are provided herein for simplicity and have not necessarily been rendered according to any particular sequence or embodiment.

FIG. 1A illustrates a perspective view of an ablation system having a monopolar electrode.

FIG. 1B illustrates a perspective view of an ablation system having a bipolar electrode.

FIG. 2 illustrates a perspective view of an ablation system having a bipolar probe that is coupled to an energy source that is capable of generating IRE energy and is inserted into target tissue of a schematically-shown organ such as a liver.

FIG. 3 illustrates a plan view of one embodiment of a power or energy source.

FIG. 4A illustrates a perspective view of the ablation system of FIG. 2 in which the bipolar probe is being withdrawn through a tissue track.

FIG. 4B illustrates a partial enlarged view of at least a portion of the probe of FIG. 4A being withdrawn along a tissue track where the probe is withdrawn farther in FIG. 4B than in FIG. 4A.

FIG. 5A is a flowchart illustrating a method of treatment using a manual switching from an IRE energy source to a thermal energy source.

FIG. 5B is a flowchart illustrating a method of treatment using an automated switching from an IRE energy source to a thermal energy source.

FIG. 6 shows a waveform including a depiction of a DC current indicating how voltage and duration of pulse can be changed for different treatment effects.

### DETAILED DESCRIPTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a tube segment” can include two or more such tube segments unless the context indicates otherwise. The term “plurality,” as used herein refers to two or more.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where the event or circumstance occurs and instances where it does not.

The term “distal” is understood to mean away from a medical practitioner and towards the body site at which the procedure is performed, and “proximal” means towards the medical practitioner and away from the body site.

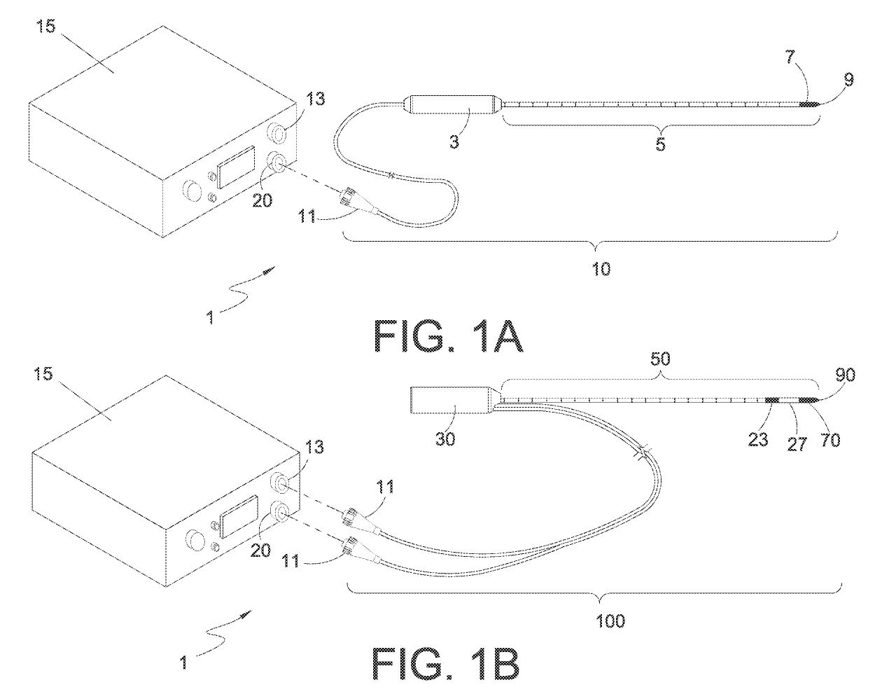

Referring now in detail to the drawings, in which like reference numerals indicate like parts or elements throughout the several views, in various embodiments, and referring to FIGS. 1-6, presented herein is an exemplary system and method for treating tumor tissue using a combination of IRE and thermal ablation.

Referring to FIG. 1A, one embodiment of an energy delivery system 1 for selectively ablating tissue is illustrated. In one aspect, the system 1 can comprise at least one energy delivery device, such as, but not limited to, a monopolar probe 10, and at least one energy delivery source or power source 15. In one aspect, at least a portion of the probe 10 can be configured for insertion into a patient. In one aspect, the at least one energy source 15 can further comprise at least a non-thermal energy source and a thermal energy source. In one aspect, the system 1 can comprise a mechanism for coupling the probe 10 to one desired energy source of the at least one energy source 15.

In one aspect, although a monopolar probe 10 is described herein, one of ordinary skill in the art will recognize that the energy delivery device used with the system 1 described herein can be a different type of energy delivery device, such as, but not limited to, a bipolar probe 100, as illustrated in FIG. 1B. In one aspect, the probe can be selected from a group consisting of: a monopolar electrode, a bipolar electrode, and an electrode array.

This can allow for utilization of an optimal energy delivery device for a given medical procedure. In one aspect, the monopolar probe 10 can comprise a handle 3, a needle 5 having a proximal end and a distal end, and at least one connector 11 of the probe. In one aspect, the needle 5 can comprise at least one electrode 7 that is positioned therein at the distal end of the needle 5 and a three-faced trocar tip 9 that is positioned therein at the distal end of the needle 5 at the distal most portion of the needle 5. In one aspect, the tip 9 can be a sharp tip and can be capable of piercing tissue.

In one aspect, at least one monopolar probe 10, as described above, can be used with system 1. In another aspect, although not illustrated, at least two monopolar probes 10, as described above, can be used with system 1. In one exemplary embodiment, it is contemplated that if more than one probe 10 is used in the system 1, the probes 10 can be used in various configurations, such as, but not limited to, a parallel configuration or a spiral configuration. In one aspect, if two probes 10 are used, it is contemplated that the tips 9 of each of the probes 10 can be positioned such that tip 9 of a first probe 10 cannot extend further than tip 9 of a second probe 10. In another exemplary aspect, the probes 10 can be positioned such that the distal tip 9 of a first probe 10 can be staggered in length compared to a distal tip 9 of a second probe 10. In one exemplary embodiment, if at least two probes 10 are used in the system 1, the at least two probes 10 can be spaced about 10 mm apart while inserted into tissue and can provide a voltage of up to 2000 volts. In yet another exemplary embodiment, the at least two probes 10 can be spaced about 15 mm apart and can have a voltage of up to about 2500 volts. In one exemplary embodiment, the at least two probes 10 can be spaced from each other such that they are approximately 40 mm apart while inserted into a target tissue and can provide a voltage of up to approximately 4000 volts.

In one aspect, the at least one electrode 7 of the monopolar probe 10 can be configured to be electrically coupled to and energized by energy source 15. Further, although not shown, one of ordinary skill in the art would recognize that at least one grounding pad can be used in conjunction with the at least one electrode 7 to complete an electrical circuit. Although a single electrode configuration is described herein, it is contemplated that other various needle and/or electrode array formations could be used in any of the embodiments described herein. An array herein refers to an orderly arrangement of multiple probes. In one aspect, this array could be a plurality or series of monopolar and/or bipolar probes arranged in various shapes, configurations, or combinations in order to allow for the ablation of multiple shapes and sizes of target regions of tissue. Various array patterns can reduce the need to reposition the electrode array during treatment by allowing multiple selectively activatable electrode patterns. In one aspect, the electrodes can be of different sizes and shapes, such as, but not limited to, square, oval, rectangular, circular or other shapes. In one aspect, the electrodes described herein can be made of various materials known in the art.

In one aspect, the electrodes described herein can be exposed up to various lengths. In one aspect, the electrodes can have an exposed length of up to approximately 20 mm while inserted into tissue, such as in the case where the at least two probes 10 are spaced up to approximately 10 mm apart. In another exemplary aspect, the electrodes can have an exposed electrode length of up to approximately 20 mm, such as in the case where the at least two probes 10 are spaced approximately 40 mm apart. In yet another aspect, the electrodes can be spaced at various distances from one another. In one aspect, the electrodes can be spaced apart a distance of from about 0.5 cm to about to 10 cm. In another exemplary embodiment, the electrodes can be spaced apart a distance of from about 1 cm to about 5 cm. In yet another embodiment, the electrodes can be spaced apart a distance of between about 2 cm and about 3 cm. In one exemplary aspect the electrode surface area can vary. In one exemplary embodiment, the electrode surface area can vary from about 0.1 cm² to about 5 cm². In yet another exemplary embodiment, the electrodes can have a surface area of between about 1 cm² to about 2 cm².

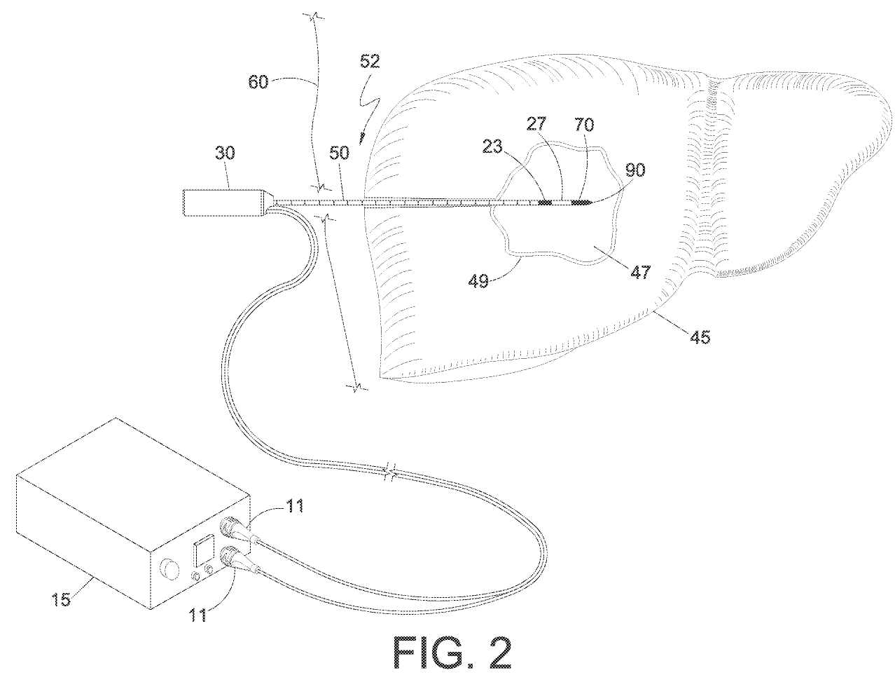

In one aspect, the system 1 can comprise a means for selectively energizing a desired energy source to ablate at least a portion of the tissue adjacent to the at least one probe 10. In one aspect, the non-thermal energy source of the at least one energy source can be selectively energized to apply non-thermal energy to at least a portion of the desired tissue region to ablate at least a portion of the desired tissue region. Thus, in one aspect, the energy source 15 can be configured to deliver non-thermal energy, such as, but not limited to, irreversible electroporation (IRE) energy to target tissue. In one exemplary embodiment, the thermal energy source can be an RF energy source. In one aspect, although not shown, during use of the system 1, the at least one probe 10 can be selectively coupled to the non-thermal energy source, and the non-thermal energy source can be selectively energized to apply non-thermal energy from the non-thermal energy source to at least a portion of the desired tissue region to ablate at least a portion of the desired tissue region In one exemplary aspect, the at least one energy source 15 can have at least one connector that is configured for selective coupling to the at least one probe 10. In one aspect, the energy source 15 can have a positive connector 20 and a negative connector 13. More particularly, the at least one connector 11 of the probe 10 can be connected to the energy source 15 via at least one of the positive connector 20 and the negative connector 13, as illustrated in FIG. 2.

In one exemplary embodiment, the power source or energy source 15 can be a RITA® model 1500× electrosurgical radiofrequency generator capable of delivering up to 250 watts of RF power. One of ordinary skill in the art would recognize that a variety of generator models could be used with the system 1 described herein. In one aspect, the generator can be powered by a battery. In one aspect, the generator 15 can be connected to a standard wall outlet that is capable of producing about 110 volts or about 230 volts. In one aspect, the power supply can be capable of being manually adjusted, depending on the voltage. In one exemplary embodiment, the generator 15 can be capable of producing a minimum voltage of about 100 volts to about 3000 volts. In one aspect, at least one of the power outlets, generators, and battery sources described herein can be used to provide voltage to the target tissue during treatment. In yet another exemplary embodiment, to achieve IRE ablation of the target region of tissue 47, the power source or generator 15 can be used to deliver IRE energy to target tissue 47, including target tissue that can be somewhat difficult to reach. In one aspect, an exemplary embodiment of an IRE generator can include anywhere from 2 to 6 positive and negative connectors, though one of ordinary skill in the art would understand that other numbers of positive and negative connectors and different embodiments of connectors could be used and may be and necessary for optimal ablation configurations.

FIG. 1B illustrates a system 1 in which a bipolar probe 100 is used, such as that described in U.S. patent application Ser. No. 12/437,843, which application is incorporated herein by reference. In one aspect, the bipolar probe 100 can comprise a handle 30, needle 50 having a proximal end and a distal end, and at least one probe connector 11. In one aspect, the needle 50 can comprise at least one electrode 70 that is positioned therein at the distal end of the needle 50 and a three-faced trocar tip 90 that is positioned at a distal most portion of a distal end of needle 50. In one aspect, the needle 50 can further comprise a first electrode 70 that is positioned at the distal most portion of the needle 50, a second electrode 23 that is positioned proximal of the electrode 70, and at least one spacer 27 that can be positioned between and adjacent to at least a portion of each of the first electrode 70 and the second electrode 23. In one aspect, at least a portion of a distal portion of the second electrode 23 can abut at least a proximal portion of spacer 27 and at least a distal portion of spacer 27 can abut at least a portion of a proximal portion of the first electrode 70. In one aspect, similar to monopolar probe 10, the bipolar probe 100 can be coupled to a thermal energy source 15. During use of the system 1, the probe 100 can be coupled to the energy source 15. More particularly, in one exemplary aspect, at least one connector 11 of the probe 100 can be connected to the energy source 15 via at least one of the positive connector 20 and the negative connector 13, as also described above.

FIG. 2 illustrates a perspective view of an ablation system having a bipolar probe that is coupled to an energy source that is capable of generating IRE energy and is inserted into target tissue of a schematically-shown organ such as a liver. Shown is the energy delivery system 1 including a bipolar probe with a handle 30, needle 50, the at least one electrode 70, a second electrode 23, the at least one spacer 27, and a three-faced trocar tip 90. Also shown is an energy delivery or power system 15, and connectors 11 to connect the probe to the power source. Also shown is a liver 45, a skin surface 60, the interstitial space 52, the target region of tissue 47 in the liver and an additional area that is an outer edge 49 of the target region of tissue 47.

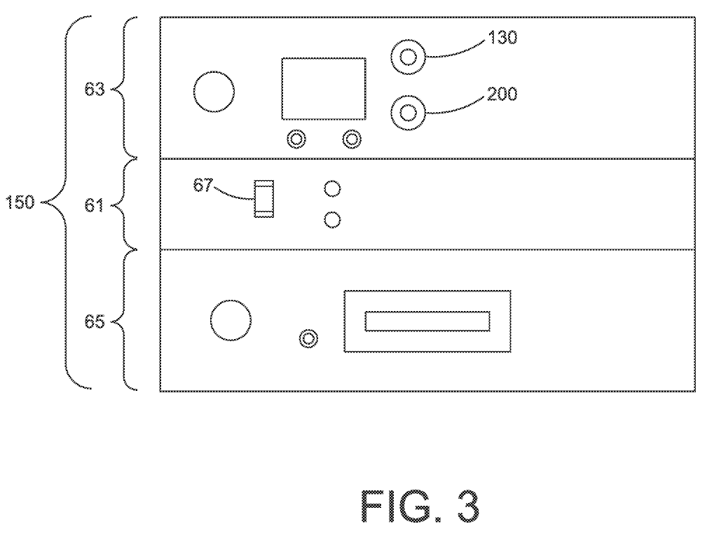

FIG. 3 illustrates an exemplary embodiment of a single power source 150 that can be used in the ablation system 1 described herein. In this embodiment the energy source of the system can be capable of providing at least two energy sources, such as, but not limited to, thermal energy or non-thermal energy. In one aspect, the non-thermal energy source and the thermal energy source can comprise a selectively configurable generator that can release energy so as to act as either or both of the non-thermal energy source and the thermal energy source. Thus, in one exemplary aspect, the same power source or generator 150 can be reconfigured to release energy for non-thermal treatment and for thermal treatment. In one aspect the single power source 150 can also comprise a switching mechanism, at least one switch 67, at least one connector, and an intermediate switching unit 61. The system can also comprise a means for selectively energizing the thermal energy source of the at least one energy source during the withdrawal of the at least one probe to thermally ablate tissue substantially adjacent to a probe track. In one aspect, the means for selectively coupling the probe to one desired energy source can comprise a means for selectively switching between the non-thermal energy source and the thermal energy source.

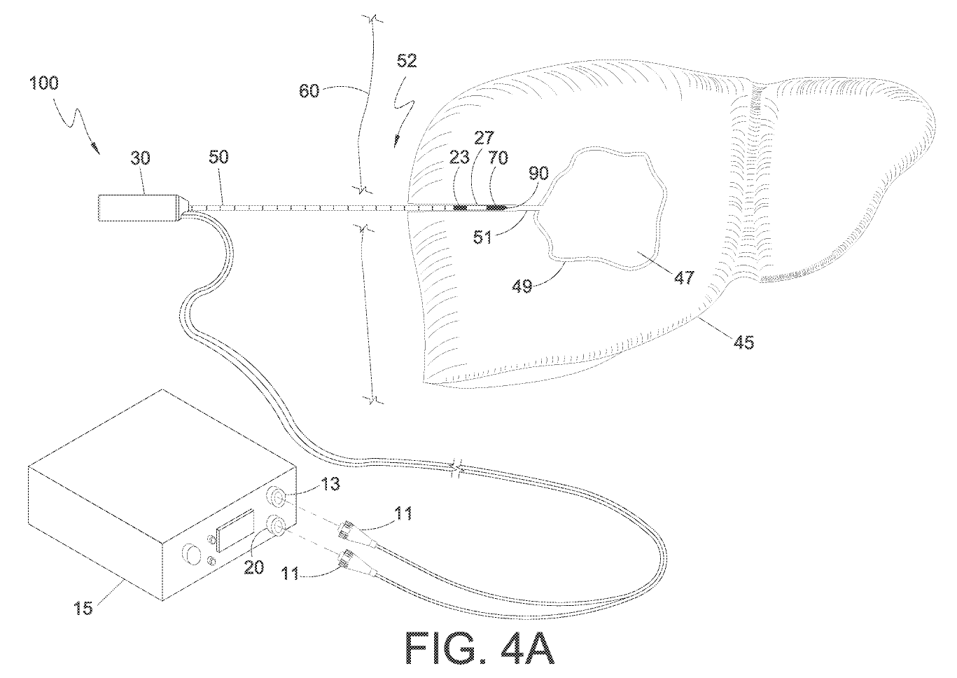

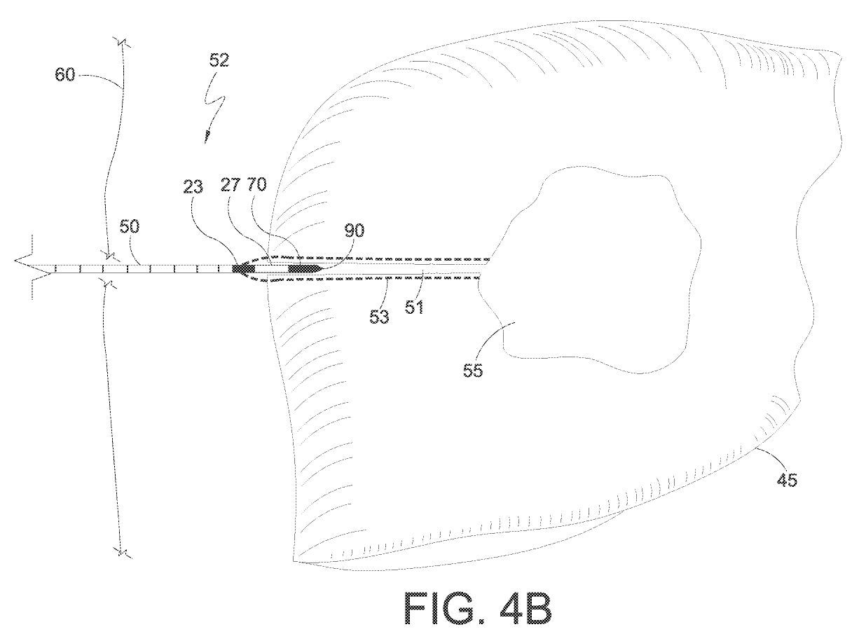

In one exemplary aspect, switch 67 can allow for automated switching between a non-thermal energy source, such as, but not limited to, IRE energy, and a thermal energy source. In one exemplary embodiment, power source 150 can have a positive connector 130 and a negative connector 200. In one aspect, the intermediate switching unit 61 allows switching between IRE energy source 63 and the thermal energy source 65. In one aspect, switch 67 can be activated to change the type of energy that is supplied to the at least one energy delivery device or probe 100. In one aspect, other power source configurations can include multiple positive and negative connectors that can be used with monopolar probes 10 or arrays where there are from 2 to 6 connectors (positive and negative) or more, allowing for multiple ablation shapes depending upon the number of probes utilized, as well as the placement position and the exposed lengths of the energy delivery devices or probes. In one aspect, the switch 67 can allow for switching from IRE energy to thermal energy as well as thermal energy to IRE energy. In one aspect, the mechanism for switching from IRE to thermal energy and back can include, but is not limited to, a switch, a toggle, or other mechanical or electrical devices known in the art, such as a button. In another embodiment, the switch 67 can be coupled to the energy delivery device or probe. In one aspect, the switch 67 can be positioned directly on the energy delivery device or probe so as to allow power switching using the same hand that is used to manipulate the probe. FIG. 4A shows an ablation system wherein bipolar probe 100 is involved in a method of selectively ablating tissue described herein. In one aspect, at least one connector 11 of bipolar probe 100 is coupled to energy source 15. Although bipolar probe 100 is described herein in the method of use, one of ordinary skill in the art would recognize that a monopolar probe could also be used in the method of use described herein. In one aspect, during the method of using the system, at least a portion of bipolar probe 100 is inserted into target tissue 47 that is located within a target organ, such as, but not limited to a liver 45. In one aspect, the target tissue 47 can comprise diseased tissue, such as, but not limited to, hepatocellular carcinoma tissue and metastatic liver cancer tissue. The system and method described herein is advantageous in that it allows for treatment of a multitude of tissues and conditions that are in some cases either inoperable through conventional surgical methods or where such surgery is contraindicated due to the status of the tissue or condition, or due to other factors related to the patient or procedure. In one aspect, as described above, energy source 15 can be configured for delivery of IRE energy to ablate target tissue 47. In one aspect, after the energy delivery device is inserted through the skin 60 into a patient's target tissue 47, as described above, the energy delivery device or probe 100 can then be used to deliver energy to the tissue 47 in order to ablate unwanted or diseased tissue (the position of the probe during target tissue ablation is shown in FIG. 2). To achieve IRE ablation of target tissue 47, standard power outlets, such as, but not limited to, can be coupled to the energy delivery device to provide energy to the system. One of ordinary skill in the art would recognize that other types of power outlets known in the art can also be used. In one aspect, any of the power outlets, generators, or battery sources described herein can provide voltage to the target tissue 47. Such voltage can be provided to tissue in the range of from about 90 volts to about 230 volts. In another exemplary embodiment such voltage can be provided to target tissue 47 at 50 volt intervals. In another exemplary aspect, such voltage can be provided to target tissue 47 in the range of from about 90 volts to about 230 volts. In other exemplary embodiments the voltage can be provided to tissue at approximately 50 volt intervals. In yet another exemplary embodiment, the IRE power source or generator 15 can be coupled to a standard wall outlet of about 110 volts or about 230 volts with a manually adjustable power supply, depending on the voltage. In another exemplary embodiment the generator 15 can have a minimum voltage of about 100 volts to about 3000 volts and can be adjustable at approximately 100 volt intervals. In one exemplary embodiment, the generator 15 can be programmable so as to operate between about 2 amps and about 50 amps. Other tests ranges can involve a lower maximum when appropriate.

In one aspect, IRE tissue ablation can be performed with variations such as those described in U.S. patent application Ser. No. 10/571,162, which application is incorporated herein by reference. In one aspect, various parameters, such as voltage, current, pulse number, pulse duration, and the dwells between pulses as can be adjusted to achieve desired treatment outcomes during ablation including IRE ablation (the dwells between pulses can be, in certain embodiments, from approximately zero to 250 microseconds, and in other embodiments can be up to a second in length, and the dwell between two specific pulses can be of the same or different length as the dwell between the two prior pulses or the two subsequent pulses). Parameters can also include position or placement of a probe or probes.

Depending on various parameters, such as voltage (including application of DC or AC or both as well as voltage per square centimeter), current, pulse number, pulse duration, and the dwell between pulses applied to tissue, the tissue can be subjected to reversible electroporation, irreversible electroporation, or thermal damage (generally considered to be resistive heating). Nonthermal IRE ablation involves ablation where the primary method of cellular disruption leading to death is mediated via electroporation (rather than factors such as effects of or responses to heating). In certain embodiments, depending on the parameters mentioned (including time that the resulting temperature occurs), cellular death can be mediated via nonthermal IRE up to approximately 50° C. In certain embodiments cellular damage from thermal heating occurs above approximately 50° C. In various embodiments, the parameters resulting in nonthermal IRE can be changed to result in the death of cells via thermal heating. The parameters can also be changed to from one having nonthermal IRE effects to alternative settings where the changed parameters also have nonthermal IRE effects.

More particularly, in one aspect, the total number of pulses and pulse trains in various embodiments can be varied based on the desired treatment outcome and the effectiveness of the treatment for a given tissue. During delivery of non-thermal IRE energy to target tissue, a voltage can be generated that is configured to successfully ablate tissue. In one aspect, certain embodiments can involve pulses between about 5 microseconds and about 62,000 milliseconds, while others can involve pulses of about 75 microseconds and about 20,000 milliseconds. In yet another embodiment, the ablation pulse applied to the target tissue 47 can be between about 20 microseconds and 100 microseconds. In one aspect, the at least one energy source can be configured to release at least one pulse of energy for between about 100 microseconds to about 100 seconds and can be adjustable at 10 microsecond intervals. In certain embodiments the electrodes described herein can provide a voltage of about 100 volts per centimeter (V/cm) to about 7,000 V/cm to the target tissue 47. In other exemplary embodiments, the voltage can be about 200 V/cm to about 2000 V/cm as well as from about 300 V/cm to about 1000 V/cm. Other exemplary embodiments can involve voltages of about 2,000 V/cm to about 20,000 V/cm. In one exemplary aspect, the bipolar probe 100 can be used at a voltage of up to about 2700 volts.

In one aspect, the number of pulses that can be used in IRE ablation can vary. In certain exemplary embodiments the number of pulses can be from about 1 pulse to about 15 pulses. In other exemplary embodiments, groups of about 1 pulse to about 15 pulses can be applied in succession following a gap of time between each pulse group or pulse train. In one exemplary embodiment the gap of time between groups of pulses can be about 0.5 second to about 10 seconds. In one aspect, pulses can be delivered to target tissue 47 using energy delivery devices, such as, but not limited to, probes, needles, and electrodes. In one aspect, such energy delivery devices can be of varying lengths suitable for use in procedures such as, but not limited to, percutaneous, laparoscopic, and open surgical procedures. In one aspect, the at least one energy source can be configured to release at least one pulse of energy for between about 100 microseconds to about 100 seconds. In one exemplary aspect, the voltage described herein can be applied using the bipolar probe 100 in pulses of 100 microseconds in length to a target region of tissue. In one aspect, the voltage can be applied in pulses of about 90 microseconds in groups of pulses or pulse-trains of 10, with an interval between pulses of about 250 milliseconds and a time between pulse-trains of about 2 seconds.

In one exemplary aspect, at least two monopolar probes 10 can be used to ablate target tissue, such that a zone of ablated tissue is produced that is approximately 22 mm×18 mm×12 mm. In one exemplary embodiment, two single probes 10 can be configured so as to involve other ablation areas, including, but not limited to, an ablation area of approximately 30 mm×25 mm×17 mm. One of ordinary skill in the art would be understood that the ablation size and shape can be advantageously varied with placement of the probes 10 and various probe types. In one aspect, during treatment, an additional area surrounding an outer edge 49 of the target region of tissue 47 is also ablated (ablation of unwanted or diseased tissue is shown in FIG. 2). This surrounding area of tissue 49 can be ablated in order to ensure patient safety and the complete and adequate ablation of the target region of tissue 47. In one aspect, during the method of use, the tri-faced trocar tip 90 of the probe 100 is used to puncture a patient's skin surface 60. In one aspect, the tip 90 and at least a portion of the probe 100 is then advanced into interstitial space 52, the tissue space between organs, and further into target tissue 47. One of ordinary skill in the art would recognize that the target region of tissue 47 can be any tissue from any organ where ablation can be used to ablate unwanted or diseased tissue, such as, but not limited to, digestive, skeletal, muscular, nervous, endocrine, circulatory, reproductive, integumentary, lymphatic, urinary tissue or organs, or other soft tissue or organs where selective ablation is desired. Soft tissue can include, but is not limited to, any tissue surrounding, supporting, or connecting other body structures and/or organs. For example, soft tissue can include muscles, tendons, ligaments, fascia, joint capsules, and other tissue. More specifically, target tissue 47 can include, but is not limited to, areas of the prostate (including cancerous prostate tissue), the kidney (including renal cell carcinoma tissue), as well as breast, lung, pancreas, uterus, and brain tissue, among others.

FIG. 4A illustrates bipolar probe 100 coupled to energy source 15. In this aspect, the energy source 15 can be a thermal energy source. In one aspect, the non-thermal energy source can be selectively energizing for a desired period of time. More particularly, the period of time can be a predetermined period of time. In yet another aspect, the period of time can be a plurality of predetermined periods of time. In one aspect, the thermal energy source is selected from the group consisting of radiofrequency (RF), focused ultrasound, microwave, lasers, thermal electric heating, traditional heating methods with electrodes using DC or AC currents, and the application of heated fluids and cold therapies (such as cryosurgery). RF energy is known in the art for effective use in tumor ablation, though it is clear that any form of temperature-mediated continuous ablation could be used at settings known the art. In one aspect, after the energy delivery device is inserted into target tissue 47, tissue is ablated, and the energy delivery device is withdrawn through a tissue track 51, as illustrated. In one aspect the thermal energy source can be an alternating current thermal energy source. In yet another aspect, the thermal energy source is a direct current thermal energy source.

In one aspect, the probe track 51 can start at the point of non-thermal ablation of the target region 47. In one aspect, thermal ablation can be initiated at the start of the probe track 51 (the start of the probe track 51 can be seen in FIGS. 2, 4A, and 4B), which in one embodiment is applied to prevent tumor seeding. As the energy delivery device or probe 100 is withdrawn, thermal energy can be applied through the probe 100 to the target tissue 47. In one aspect, the probe is selectively energized with thermal energy to ablate tissue proximate a distal end of the probe track and proximate to a boundary of the tissue ablated by the non-thermal energy source.

In one aspect, IRE treatment of target tissue 47, followed by thermal ablation of at least one tissue track 51 can be performed during procedures such as, but not limited to, laparoscopic procedures and open surgical procedures. In one aspect track 51 can be ablated during removal or repositioning of a probe 100 from a target region of tissue 47, as well as through a portion of the interstitial space 52. FIG. 4B illustrates an enlarged portion of FIG. 4A after the target tissue is treated with IRE energy and where the probe has been farther withdrawn in FIG. 4B than in FIG. 4A. In one aspect, after delivery of IRE energy to the target tissue, an ablated region 55 of tissue remains. In one aspect, ablated region 55 of tissue includes target tissue region 47 and the surrounding area of tissue 49 shown in FIG. 4A. In one exemplary embodiment, after treatment of the target tissue using IRE, treatment parameters can be reset to bring about thermal track ablation. In one aspect, after IRE treatment of the target tissue, the energy delivery device or probe is withdrawn through a tissue track 51. In one aspect, upon withdrawal of the energy delivery device or probe 100 (and in some cases repositioning) of the energy delivery device through tissue track 51 to ablate tissue, a tissue track is coagulated, and tumor cell seeding can be prevented. In one aspect thermal energy, such as, but not limited to RF energy, can be applied to the ablation track 51 during probe withdrawal such that a track ablation zone 53 is created. In one aspect, the track ablation zone 53 can be defined by a thin layer of tissue immediately surrounding the probe track 51 that has been ablated. In one aspect, the track ablation zone 53 is created in order to define a cauterized zone and to prevent seeding as the probe 100 is withdrawn from the tissue track 51. In another aspect the track ablation zone 53 is created to stop bleeding. It is important to prevent track seeding during probe withdrawal, especially during procedures that could involve excess bleeding, such as those involving tumor ablation of at least a portion of organs such as, but not limited to, the liver 45, which is a highly vascularized organ. In one aspect, the track ablation zone 53 can extend from the total IRE ablation region 55, through a portion of the liver 45 into the interstitial space 52. In one aspect the track ablation zone 53 can extend outside of the liver 45 and into the interstitial space 52 but does not extend to skin surface 60 since application of thermal energy close to the skin surface can cause complications for patients.

In one aspect, the generator 15 used during the thermal track ablation procedure can be configured to have various track ablation settings and capabilities. In one exemplary aspect, the RITA® 1500× generator described above can be used as an RF energy source. In one aspect, the RF energy source can be used to ablate track tissue using 25-50 watts of power. In other exemplary aspects, one of ordinary skill in the art would recognize that smaller or larger amounts of power can be used in various embodiments, as necessary, in order to provide track ablation. In one exemplary embodiment utilizing the 1500× generator, the RF power source can provide AC power in addition to being used for track ablation, while the IRE power source can be used to provide DC power.

In one aspect, if a thermal energy source is used, it could be used with a variety of techniques to bring about tissue ablation. In one exemplary aspect, additional embodiments can involve track ablation performed using one or more of radiofrequency (RF), focused ultrasound, microwaves, lasers, thermal electric heating, traditional heating methods with electrodes using DC or AC currents, and application of heated fluids and cold therapies, such as, but not limited to, that used in cryosurgery. In one aspect h heat energy can be delivered in certain embodiments via pulses that can be in a range of about 100 microseconds to about 10 seconds. In other exemplary embodiments the at least one energy source can be configured to release or deliver at least one pulse of heat energy in a range of about 100 microseconds to about 1 second. In yet another exemplary embodiment, at least one energy source can release or deliver at least one pulse of energy for between about 100 microseconds to about 1000 microseconds. In yet another exemplary embodiment, at least one pulse can be delivered in a range of from about 1 microsecond to about 100 microseconds.

In one exemplary embodiment thermal energy can be applied such that it produces fluctuations in temperature to effect treatment. In one aspect, the thermal energy provided to the tissue can heat the target tissue to between about 50° C. and about 105° C. to bring about cell death. In one aspect the temperature can be adjusted such that it can be lesser or greater than this temperature range, depending on the exact rate of speed of removal of the energy delivery device from the target tissue 47. In one embodiment the temperature used is between about 105° C. and about 110° C., although one of ordinary skill would recognize that temperatures above about 105° C. can cause tissue vaporization. Ellis L, Curley S, Tanabe K. Radiofrequency Ablation for Cancer; Current Indications, Techniques, and Outcomes, N.Y.: Springer, 2004. In one exemplary embodiment, thermal energy can be used to ablate approximately 2-3 mm of tissue surrounding track 51. In one aspect this tissue thickness can be varied depending upon various factors, such as, but not limited to, the condition of the target tissue 47, the various parameters used, and the treatment options.

In one embodiment the mechanisms through which the user sets the parameters for bringing about IRE effects are changed to bring about thermal results through thermal heating that is resistive heating. In certain embodiments the mechanisms are reset such that DC energy is applied to bring about thermal track ablation. In one exemplary embodiment, track ablation can be performed using DC current. In one aspect, the DC current can be used for heating the target tissue 47 in the track 51. In one aspect, at least one pulse of DC current can be delivered in one direction. In yet another aspect, at least one pulse of DC current can be delivered from the opposite direction of an electrical circuit. In one aspect, DC current can be applied such that the temperature of the tissue 47 can be between about 42° C. and about 110° C. In one aspect, the DC current can be applied such that thermal damage is induced at a temperature as low as about 42° C. In yet another aspect, as the rate of probe removal increases, the DC current can be applied to the target tissue 47 such that the temperature can be from about 50° C. to about 60° C. Davalos R, Mir L, Rubinsky B. Tissue Ablation with Irreversible Electroporation. Annals of Biomedical Engineering, Vol. 33(2):223-231 (2005). One of ordinary skill in the art would recognize that various lengths of DC pulses can be applied to bring about effective track ablation. In yet other embodiments, AC pluses can be applied as the energy delivery device is removed from the target tissue 47 in stages.

In summary, the method for selectively ablating tissue involves providing at least one energy source, such as a generator, described above. In one aspect, the at least one energy source can comprise at least a non-thermal energy source and a thermal energy source, providing at least one probe that is configured to be selectively manually operatively coupled to a desired energy source of the at least one energy source, positioning, via a probe track, at least a portion of the at least one probe within a desired region of a target tissue. In one aspect, the selective coupling of the probe to the thermal energy source comprises the actuating a switch to operatively select between the non-thermal energy source and the thermal energy source. Then at least one probe is selectively coupled to the non-thermal energy source, and the non-thermal energy source is selectively energized to apply non-thermal energy from the non-thermal energy source to at least a portion of the desired region to ablate at least a portion of the desired region, selectively coupling the at least one probe to the thermal energy source, withdrawing the at least probe from the desired region, and selectively energizing the thermal energy source to apply thermal energy during at least a portion of withdrawal of the at least one probe to ablate tissue substantially adjacent to the probe track. In one aspect, prior to selectively coupling the at least one probe to the thermal energy source, the at least one probe is operatively decoupled from the non-thermal energy source.

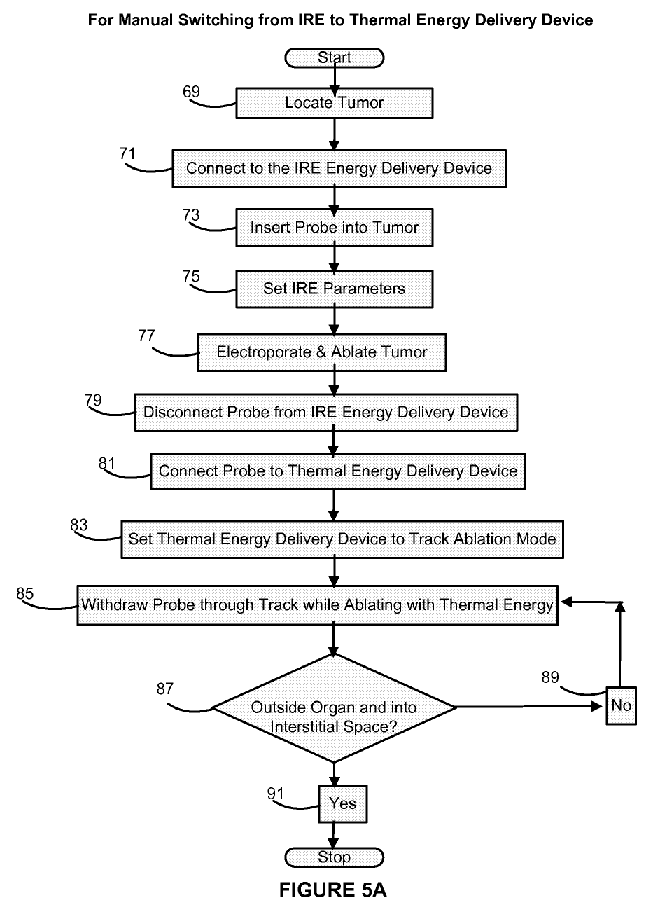

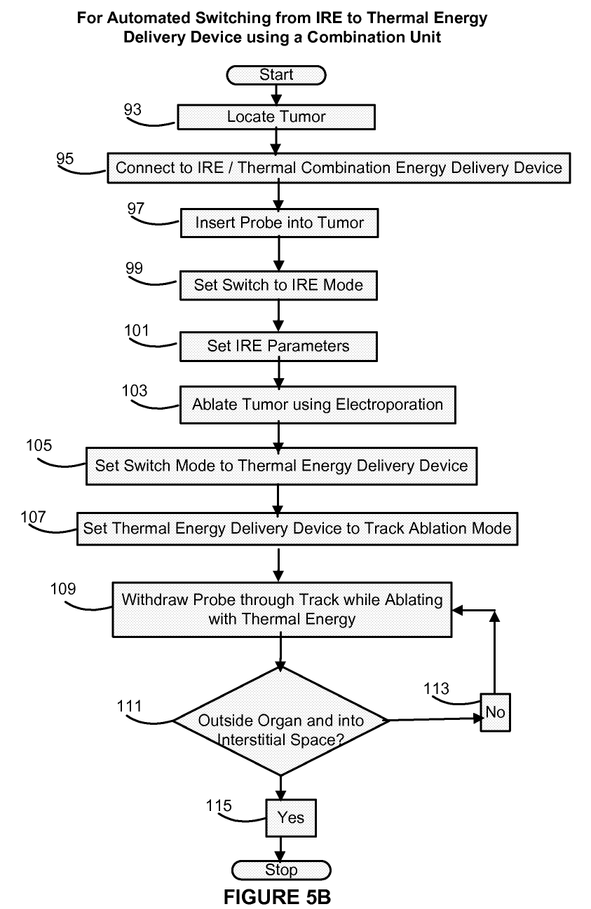

FIG. 5A and FIG. 5B illustrate a flowchart of the method for treating target tissue 47 using an IRE energy source and a thermal energy source, respectively. In one aspect, treatment can be alternated between IRE energy and thermal energy using a manual switch, as depicted in FIG. 5A. The method is shown from the start to stop point of FIG. 5A as acts 69 to 91. In the manual switching procedure, a physician locates 69 a target region of tissue 47 such as a tumor using technology known in the art such as ultrasound imaging. In one aspect an energy delivery device or probe is connected 71 to IRE energy source, and the energy delivery device or probe is then inserted 73 into at least a portion of target region of tissue 47 such as a tumor. In one aspect, the IRE parameters are set 75 to the desired settings, and the target region of tissue 47 can be ablated 77 using an IRE process that is precise and that spares surrounding bile ducts, blood vessel, and connective tissue. The probe is then manually disconnected 79 from the IRE energy source, and the probe is connected 81 to a thermal energy source, where a track ablation mode can be set 83 for ablation of the tissue adjacent to the probe as the probe is withdrawn through the probe track 85 and from the organ and into the interstitial space in a direction toward the skin. In one aspect, this method provides the advantage of a continuous ablation of the tissue 47 as the probe 10/100 is removed. Ablation can be continued as the probe 10/100 is withdrawn. After step 85, a user can evaluate whether the probe 10/100 has been withdrawn from the target tissue region 47 and is outside 87 the organ and into the interstitial space 52. If the probe has not been withdrawn from the organ and has not reached 89 the interstitial space 52, ablation will continue, though after the probe 10/100 has been withdrawn 91 into the interstitial space 52, ablation will be stopped; certain embodiments include stopping points between the organ exit site to the skin surface.

Still referring to FIG. 5A, although the method described involves the ablation of at least part of a target tissue region 47 within an organ, one of ordinary skill in the art will recognize that in some embodiments the probe 10/100 can be withdrawn directly from a tissue 47, such as, but not limited to, a tumor not associated with an organ. In yet another aspect, the probe 10/100 can be withdrawn directly from an organ following ablation of at least a part of the organ. In each of these embodiments, track ablation continues until a point is reached in the interstitial space before reaching the skin surface. In one aspect, either the ablation is carried out directly from the target tissue region 47 and into the interstitial space 52, where the ablation can cease, or the track ablation can be from the target tissue region 47 through at least part of an organ and into the interstitial space 52, where tissue ablation stops prior to reaching the skin.

FIG. 5B is a flowchart illustrating a method of automatic switching from an IRE energy source to a thermal energy source utilizing switching in a combination thermal/non-thermal unit (93-115). Shown is that the tumor is located 93, a connection is made to the IRE/thermal combination energy delivery device 95, the probe is inserted into tissue such as a tumor 97, the mode is set to IRE 99 and the IRE parameters are set 101. Once the tissue such as a tumor is ablated 103, the mode can be set to thermal 105, wherein the device can be used in certain embodiments to ablate tissue (to supplement the IRE ablation that was performed). The mode can be set to track ablation mode 107. The probe is withdrawn while ablating with thermal energy 109, and ablation is stopped once the ideal point is reached outside the organ or tumor or target region (depending on question 111 and answers 113, 115)

In one aspect the switching outlined in FIG. 5B differs from the switching of FIG. 5A in that instead of manual switching between IRE and thermal energy sources, ablation is automatic switched between IRE energy and thermal energy. In one aspect, this embodiment can involve the use of a switch. In one embodiment, the combination unit can include pre-set track ablation settings. In one aspect, the pre-set track ablation settings can be chosen through the push of a button or switch. In another exemplary embodiment, a track ablation mode can be chosen in which settings can be adjusted. One of ordinary skill in the art would understand that other exemplary methods can include switching from an IRE energy source to a thermal energy source or from a thermal energy source to an IRE energy source, and that this switching can be performed more than once. In one aspect, he switch can be thrown multiple times as necessary in a given procedure. One of ordinary skill in the art will recognize that the combined method of IRE ablation of target regions of tissue 47 and thermal track ablation can be used with other devices and procedures. Switching between non-thermal or IRE energy and thermal energy is advantageous because it allows non-thermal focal ablation of target tumor tissue 47 with tissue sparing and provides for continuous thermal track ablation to prevent or eliminate problems such as seeding and coagulation as the probe 10/100 is withdrawn through tissue track 51. In one exemplary aspect this combination non-thermal/thermal ablation can be performed with a single probe 10/100. The use of a single probe is advantageous because it allows for fewer puncture sites, shortens and simplifies the treatment procedure, and causes fewer traumas to the patient.

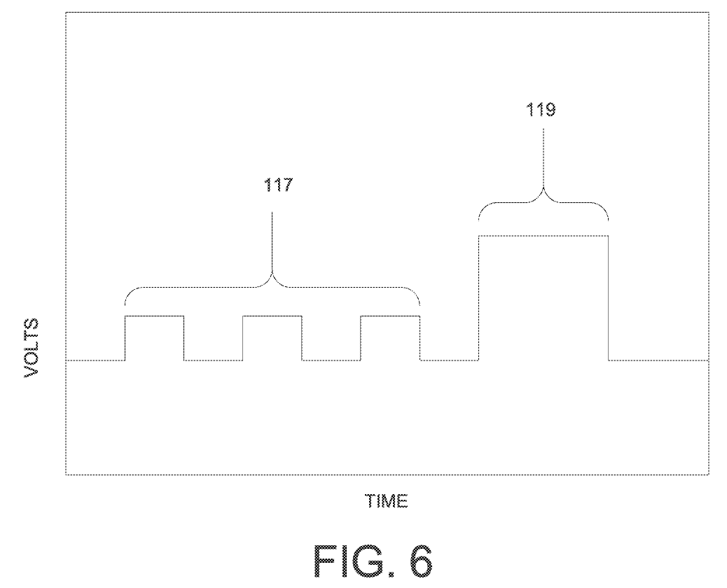

FIG. 6 shows a waveform including a depiction of a DC current indicating how voltage and duration of pulse can be changed for different treatment effects. More specifically, three pulses (117) are shown that are of equal voltage (shown in the equivalent of a Y-axis in FIG. 6) and duration to one another (that can be seen by looking at the pulse in relation to the equivalent of an X-axis for time in FIG. 6), and a fourth pulse (119) is also shown that is of greater voltage and greater duration than the previous three pulses (117). This depiction indicates that in certain embodiments target tissue can be ablated using a setting such as 117 to produce IRE nonthermally, and that the DC voltage can be increased and the duration of the pulse increased to cause a certain effect on tissue; in various embodiments the target tissue would be ablated by application of pulses 117, and a single pulse or multiple pulses of greater voltage as well as greater duration (or both) can be applied for part of or the entire time of withdrawal of the probe from the site of ablation to the point exiting the body where the level of voltage and duration lead to thermal effects from resistive heating for preventing tumor seeding, coagulating blood vessels, or both.

Still referring to FIG. 6, in various embodiments 119 is more than one pulse that is simply longer in duration than any of the pulses in 117 but which mediate thermal heating via resistive heating. Also, the thermal heating can be brought about by changing 119 such that the pulses are greater in number, the pulses are longer, the dwell between pulses is smaller, or the voltage is higher. It is also conceivable to alter one or both the voltage as well as pulses to increase or decrease either or both (including having the option to vary time between pulses) to bring about thermal effects for track ablation. In certain embodiments the change from pulses leading to IRE effects 117 to the pulse or pulses leading to thermal effects 119 are used to bring about IRE and thermal effects on tissue where both effects are within the target region. Also, in certain embodiments the order of application of pulses 117 and pulse or pulses 119 is switched in the target region or in the tissue track or both to most effectively treat the patient (so application of 119 could be before 117). Also, pulses 117, and pulse or pulses 119 can be used in conjunction with thermal heating methods such as Radiofrequency such that nonthermal IRE effects, effects from resistive heating due to DC current changes (such as that shown in 119), and thermal heating effects from AC current (such as RF) can be brought about in any order in target tissue or in a tissue track for the benefit of the patient. For example, a tumor mass can be treated with IRE or RF (or other AC as well as other DC pulses leading to resistive heating) or more than one of these in any order so as to ablate the target tissue or tissues and control bleeding or coagulate or ablate vessels or cells, and then upon probe removal, IRE or RF (or other AC as well as other DC pulses leading to resistive heating) pulses can be uses as necessary together or independently in any order to control bleeding, coagulate or ablate vessels, ablate tumor cells, or to ablate or treat tissue surrounding the track. In certain embodiments the changes between treatments or treatment methods can be brought about using a mechanism or device or system for altering or changing one or more parameters herein described via an energy source; the source could have one or more generators coupled and parameters could be determined using mechanisms of a system or a generator or energy source, and the mechanisms could have control components allowing user changes from a probe directly or from the energy source directly.

What is claimed is:

1. A method for selectively ablating tissue comprising:
providing at least one energy source, wherein the at least one energy source comprises at least a non-thermal energy source and a thermal energy source;
providing at least one probe that is configured to be selectively operatively coupled to a desired energy source of the at least one energy source;
positioning, via a probe track, at least a portion of the at least one probe within a desired region of a target tissue;
selectively coupling the at least one probe to the non-thermal energy source;
selectively energizing the non-thermal energy source to apply non-thermal energy from the non-thermal energy source to at least a portion of the desired region to ablate at least a portion of the desired region;
selectively coupling the at least one probe to the thermal energy source;
withdrawing the at least one probe from the desired region; and
selectively energizing the thermal energy source to apply thermal energy during at least a portion of withdrawal of the at least one probe to ablate tissue substantially adjacent to the probe track.

2. The method of claim 1, wherein the non-thermal energy source is selectively energizing for a desired period of time.

3. The method of claim 2, wherein the desired period of time is a predetermined period of time.

4. The method of claim 3, wherein the desired period of time is a plurality of predetermined periods of time.

5. The method of claim 1, further comprising, prior to selectively coupling the at least one probe to the thermal energy source, operatively decoupling the at least one probe from the non-thermal energy source.

6. The method of claim 1, wherein the steps of selectively coupling the at least one probe to the non-thermal energy source and selectively coupling the at least one probe to the thermal energy source is done manually.

7. The method of claim 1 or 6, wherein selectively coupling the at least one probe to the non-thermal energy source and selectively coupling the at least one probe to the thermal energy source comprises actuating a switch to operatively select between the non-thermal energy source and the thermal energy source.

8. The method of claim 1, wherein the thermal energy source is an alternating current thermal energy source.

9. The method of claim 1, wherein the thermal energy source is a direct current thermal energy source.

10. The method of claim 1, wherein said thermal energy source is selected from a group consisting of: radiofrequency (RE), focused ultrasound, microwaves, lasers, thermal electric heating, and cryosurgery.

11. The method of claim 1, wherein the probed is selected from a group consisting of: a monopolar electrode, a bipolar electrode, and an electrode array.

12. The method of claim 1, wherein the target tissue is selected from a group consisting of: digestive tissue, skeletal tissue, muscular tissue, nervous tissue, endocrine tissue, circulatory tissue, reproductive tissue, integumentary tissue, lymphatic tissue, urinary tissue, and soft tissue.

13. The method of claim 1, wherein the target tissue is selected from a group consisting of: liver tissue, prostate tissue, kidney tissue, lung tissue, pancreas tissue, uterus tissue, breast tissue, and brain tissue.

14. The method of claim 1, wherein the probe is selectively energized with thermal energy to ablate tissue proximate a distal end of the probe track and proximate to a boundary of the tissue ablated by the non-thermal energy source.

15. The method of claim 1, wherein at least one energy source is configured to release at least one pulse of energy for between about 100 microseconds to about 100 seconds.

16. The method of claim 1, wherein at least one energy source is configured to release at least one pulse of energy for between about 100 microseconds to about 1 second.

17. The method of claim 1, wherein at least one energy source is configured to at least one pulse of energy for between about 100 microseconds to about 1000 microseconds.

18. The method of claim 1, wherein the non-thermal energy source and the thermal energy source comprised a selectively configurable generator that can release energy so as to act as both the non-thermal energy source and the thermal energy source.

19. A method for selectively ablating tissue comprising:
providing at least one energy source, wherein the at least one energy source comprises at least a non-thermal energy source and a thermal energy source;
providing at least one probe that is configured to be selectively operatively coupled to a desired energy source of the at least one energy source;
positioning, via a probe track, at least a portion of the at least one probe within a desired region of a target tissue;
selectively coupling the at least one probe to the non-thermal energy source;
selectively energizing the non-thermal energy source to apply non-thermal energy from the non-thermal energy source to at least a portion of the desired region to ablate at least a portion of the desired region;
changing at least one parameter on the non-thermal energy source;
withdrawing the at least one probe from the desired region; and
selectively energizing the thermal energy source to apply thermal energy during at least a portion of withdrawal of the at least one probe to ablate tissue substantially adjacent to the probe track.

20. The method of claim 19 wherein the at least one parameter is selected from a group consisting of: voltage, current, pulse number, pulse duration, and a dwell between two pulses.

21. A system for selectively ablating tissue comprising:
at least one energy source, wherein the at least one energy source comprises a non-thermal energy source and a thermal energy source;
at least one probe;
means for selectively coupling the probe to one desired energy source of the at least one energy source;
means for selectively energizing the non-thermal energy source of the at least one energy source to apply non-thermal energy to at least a portion of the desired region to ablate at least a portion of the desired region; and
means for selectively energizing the thermal energy source of the at least one energy source during the withdrawal of the at least one probe to thermally ablate tissue substantially adjacent to a probe track.

22. The system of claim 21, wherein the means for selectively coupling the probe to one desired energy source comprises means for selectively switching between the non-thermal energy source and the thermal energy source.

23. A system for selectively ablating tissue comprising:
at least one energy source, wherein the at least one energy source comprises a non-thermal energy source and a thermal energy source;
at least one probe, wherein at least a portion of the probe is configured for insertion therein a patient;
means for selectively coupling the probe to one desired energy source of the at least one energy source; and
means for selectively energizing the one desired energy source to ablate at least a portion of the tissue adjacent to the at least one probe.

24. The system of claim 23, wherein the means for selectively energizing the one desired energy source comprises:
means for selectively energizing the non-thermal energy source of the at least one energy source to apply non-thermal energy to at least a portion of a desired region to ablate at least a portion of the desired region; and
means for selectively energizing the thermal energy source of the at least one energy source during the withdrawal of the at least one probe to thermally ablate tissue substantially adjacent to a probe track.

25. The system of claim 24, wherein the means for selectively coupling the probe to one desired energy source comprises means for selectively switching between the non-thermal energy source and the thermal energy source.

26. A method for selectively ablating tissue comprising:
providing at least one energy source, wherein the at least one energy source comprises at least a non-thermal energy source and a thermal energy source and at least one means for selectively adjusting at least one parameter;
providing at least one probe that is configured to be selectively operatively coupled to the at least one energy source;
positioning, via a probe track, at least a portion of the at least one probe within a desired region of a target tissue;
selectively coupling the at least one probe to the at least one energy source;
selectively adjusting the at least one parameter to deliver non-thermal energy to the target tissue; selectively energizing the at least one energy source to apply non-thermal energy from the at least one energy source to at least a portion of the desired region to ablate at least a portion of the desired region;
selectively adjusting the at least one parameter to deliver thermal energy to the target tissue;
withdrawing the at least one probe from the desired region; and
selectively energizing the at least one energy source to apply thermal energy during at least a portion of withdrawal of the at least one probe to ablate tissue substantially adjacent to the probe track.

27. The method of claim 26, wherein the at least one parameter is selected from group comprising: voltage, pulse duration, and pulse number.
